(19) United States
(12) Patent Application Publication
(10) Pub. No.: US 2022/0275436 A1
(43) Pub. Date: Sep. 1, 2022

(54) COMPOSITIONS AND METHODS FOR AMPLIFICATION OF STR LOCI

(71) Applicant: QIAGEN GMBH, Hilden (DE)

(72) Inventors: Margaretha Willuweit, Köln (DE); Stefan Otto Cornelius, Köln (DE)

(21) Appl. No.: 17/636,582

(22) PCT Filed: Aug. 13, 2020
(86) PCT No.: PCT/EP2020/072717

§ 371 Date: Feb. 18, 2022

(30) Foreign Application Priority Data
Aug. 21, 2019 (EP) 19192884.5

(51) Int. Cl. C12Q 1/6858; C12Q 1/6848; C12N 9/12

(52) U.S. Cl. CPC C12Q 1/6858; C12Q 1/6848; C12N 9/1252; C12Y 207/07007

(57) ABSTRACT

A first aspect of the invention disclosed herein is directed to a composition for performing an amplification reaction of a nucleic acid template, the composition comprising: a) a buffer, b) a DNA polymerase, c) one or more primers and d) a mixture of deoxynucleotides (dNTPs), wherein the mixture of dNTPs comprises a higher dATP concentration than that of either dGTP, dCTP or dTTP. A second aspect of the invention disclosed herein is directed to a method for amplification of a target sequence, the method comprising the steps of: a) performing a PCR amplification using the composition according to the first aspect and its embodiments of the present invention, thereby obtaining a PCR product, b) determining the presence of the target sequence in the PCR product. A third aspect of the invention disclosed herein is directed to primer or set of primers for detecting a target sequence, wherein the primer or each primer in the set of primers comprises a 5′-end G. A fourth aspect of the invention disclosed herein is directed to a kit for STR analysis.

### FIELD OF THE INVENTION

The present invention is in the field of molecular biology, diagnostics, more particularly in the field of analytical and forensic sciences. The invention is further in the field of nucleic acid amplification and encompasses a composition and a method for performing polynucleotide chain reaction (PCR).

### BACKGROUND OF THE INVENTION

Molecular biology techniques are widely used in genotyping applications and other areas such as biological research, forensic and diagnostic applications.

Forensic workflow schemes require the amplification of so called short tandem repeat (STR) markers. These markers are genetic elements of variable lengths that are characterized by short repetitive sequence motifs and are used in combination with other STR loci to obtain a genetic fingerprint of an individual.

A narrow range of input DNA from 0.5 to 2 ng is often needed to produce optimal results with for example multiplex DNA typing kits. Furthermore, the quality of standards for forensic DNA testing laboratories requires human-specific DNA quantification. This is due to isolation techniques that can recover human DNA as well as bacterial or exogenous DNA. A number of procedures have been developed to permit quantification of human-specific DNA including blotting techniques, liquid based hybridization assays and real-time polymerase chain reaction (PCR). Currently, real-time PCR is the dominant technique due to its wide dynamic range and ease of automation.

After amplification, the resulting PCR products are labelled using fluorescent dyes and the technique of capillary electrophoresis (CE) is employed to separate said amplification products according to their molecular size. The fluorescent signals are represented as peaks in the electropherogram.

Thermostable DNA polymerases can catalyze non-templated addition of a nucleotide to the 3′ end of amplification products (Smith et al. 1995, Genome Res. 5(3):312-317). Particularly, it has been observed that in PCR reactions with Taq DNA polymerase a dATP nucleotide is incorporated after amplification to the specific target sequence. As a result, the amplicon is one base longer than the original template sequence. This event, called 3′ A overhang, is not corrected by the Taq DNA polymerase because it lacks proofreading function and represents a potential source of error in genotyping studies employing Taq DNA polymerase to amplify microsatellite loci.

In STR analysis, the problem of split peak formation depends on the amount of template and the particular cycling protocol used. Generally, the amplicon obtained by PCR reactions with Taq DNA polymerase comprises products with and without 3′ A overhang. Therefore, the electropherograms of the PCR products are characterized by two closely spaced peaks which cannot be separated properly by the analysis software and thus lead to a costly post-analysis of these samples. This effect occurs more frequently especially with very high amount of DNA template.

The issue of 3′ A overhang in amplicon obtained by PCR reactions with Taq DNA polymerase has been object of study.

Magnuson reported that certain terminal nucleotides can either inhibit or enhance adenine addition by Taq and that PCR primer design can be used to modulate this activity (Magnuson et al. 1996, BioTechniques 21(4):700-709).

The effect of pool imbalances on the frameshift fidelity of HIV-1 reverse transcriptase has been also investigated by Bebenek (Bebenek et al. 1992, J. Biol. Chem. 267(6):3589-3596). However, the models developed by Bebenek do not provide a consistent explanation to all pool imbalance-mediated effects on HIV-1 reverse transcriptase frameshift fidelity.

Brownstein focused on the consensus sequences that promote or inhibit 3′ A overhang. Particularly, it has been found that modifying reverse and/or forward primers by including a suitable nucleic acid sequence is it possible to control the formation of adenylated or non-adenylated PCR product (Brownstein et al. 1996, BioTechniques 20(6):1004-1010).

In view of the limitations and drawbacks affecting current PCR amplification methods, there is a need for a rapid and reliable method for amplifying, analyzing and typing polymorphic DNA fragments, particularly minisatellite, microsatellite or STR DNA fragments. The invention disclosed herein provides a solution to the above issues.

### SUMMARY OF THE INVENTION

A first aspect of the invention disclosed herein is directed to a composition for performing an amplification reaction of a nucleic acid template, the composition comprising
a. a buffer,
b. a DNA polymerase,
c. one or more primers and
d. a mixture of deoxynucleotides (dNTPs),

wherein the mixture of dNTPs comprises a higher dATP concentration than that of either dGTP, dCTP or dTTP.

A second aspect of the invention disclosed herein is directed to a method for amplification of a target sequence, the method comprising the steps of:
a. performing a PCR amplification using the composition according to the first aspect and its embodiments of the present invention, thereby obtaining a PCR product,
b. determining the presence of the target sequence in the PCR product.

A third aspect of the invention disclosed herein is directed to a primer or set of primers for detecting a target sequence, wherein the primer or each primer in the set of primers comprises a 5′-end G.

A fourth aspect of the invention disclosed herein is directed to a kit for STR analysis, the kit comprising:
a. a mixture of dNTPs, the mixture comprising dATP, dGTP, dCTP and dTTP, wherein the concentration of dATP is higher than dGTP, dCTP and dTTP;
b. a set of primers, wherein each primer in the set of primers comprises a 5′-end G;
c. a buffer;
d. a DNA polymerase lacking 3′-5′ exonuclease activity;
e. a nucleic acid template comprising a short tandem repeat (STR) sequence.

### DESCRIPTION OF THE FIGURES

FIG. 1A shows the analytical profile of PCR amplifications using 2 ng of Human DNA template with normal dNTP concentration (0.4 mM each dNTP). The amplicon is one base longer than the original template sequence (3′ A overhang); see circled peaks.

FIG. 1B shows the analytical profile of PCR amplifications using 8 ng of Human DNA template with normal dNTP concentration (0.4 mM each dNTP) The circled peaks represent the Marker with the minus A-Peaks

FIG. 2A shows the analytical profile of PCR amplifications using 2 ng of Human DNA template with asymmetrical dNTP concentration (0.4 mM each dNTP and 0.3 mM extra dATP). The circled peaks represent the identical marker without the minus A peak from the record 1A.

FIG. 2B shows the analytical profile of PCR amplifications using 2 ng of Human DNA template with asymmetrical dNTP concentration (0.4 mM each dNTP and 0.1 mM extra dATP). The circled peaks represent the second record to show the effect with 0.1 mM dATP reduced number of minus A peaks.

FIG. 2C shows the analytical profile of PCR amplifications using 2 ng of Human DNA template with asymmetrical dNTP concentration (0.4 mM each dNTP and 0.2 mM extra dATP). The circled peaks represent third record to show the effect with 0.2 mM dATP reduced number of minus A peaks.

FIG. 2D shows the analytical profile of PCR amplifications using 2 ng of Human DNA template with asymmetrical dNTP concentration (0.4 mM each dNTP and 0.4 mM extra dATP). The circled peaks represent the record show record without minus A peak.

FIG. 3 shows the effect of the dATP titration on the split peak formation.

FIG. 4 shows the ratio of the −A peak to the full-length amplificated.

FIG. 5 shows the effect of altering the concentration of dATP in a mixture of dNTPs in PCR amplification and detection of DYS391 marker. (a) 0.4 mM each dNTPS; (b) 0.1 mM dATP extra and 0.4 mM each dNTPS; (c) 0.2 mM dATP extra and 0.4 mM each dNTPS; (d) 0.4 mM dATP extra and 0.4 mM each dNTPS.

FIG. 6 shows the effect of altering the concentration of dATP in a mixture of dNTPs in PCR amplification and detection of D10S1248 marker. (a) 0.4 mM each dNTPS; (b) 0.1 mM dATP extra and 0.4 mM each dNTPS; (c) 0.2 mM dATP extra and 0.4 mM each dNTPS; (d) 0.4 mM dATP extra and 0.4 mM each dNTPS.

FIG. 7 shows the effect of altering the dNTP amplification and detection of DYS391, D10S1248, SE33 marker concentration of A) 0.4 mM dNTP; B) 0.4 mM dNTP+0.3 mM dATP; C) 0.4 mM dNTP+0.3 mM dCTP; D) 0.4 mM dNTP+0.3 mM dGTP; E) 0.4 mM dNTP+0.3 mM dTTP.

FIG. 8 shows the effect of (A) only dNTPs having same concentration; (B) 0.4 mM dNTPs+Taq; (C) 0.4 mM dNTPs+extra 0.3 mM dATP; (D) 0.4 mM dNTPs+extra 0.3 mM dATP+Taq with the STR markers D2S441 and D18S551.

FIG. 9 shows the effect that only the excess of dATP led to the elimination of split peaks. Various concentrations were tested. (A) Control sample with equimolar dNTPs and excess of +0.3 mM of dATP or dCTP or dGTP or dTTP; (B) control sample with equimolar dNTPs or excess of +0.4 mM of dATP or dCTP or dGTP or dTTP; (C) Control sample with equimolar dNTPs or excess of +0.6 mM of dATP or dCTP or dGTP or dTTP, (D) control sample with equimolar dNTPs or excess of +1 mM of dATP or dCTP or dGTP or dTTP with the STR markers D2S441 and D18S551.

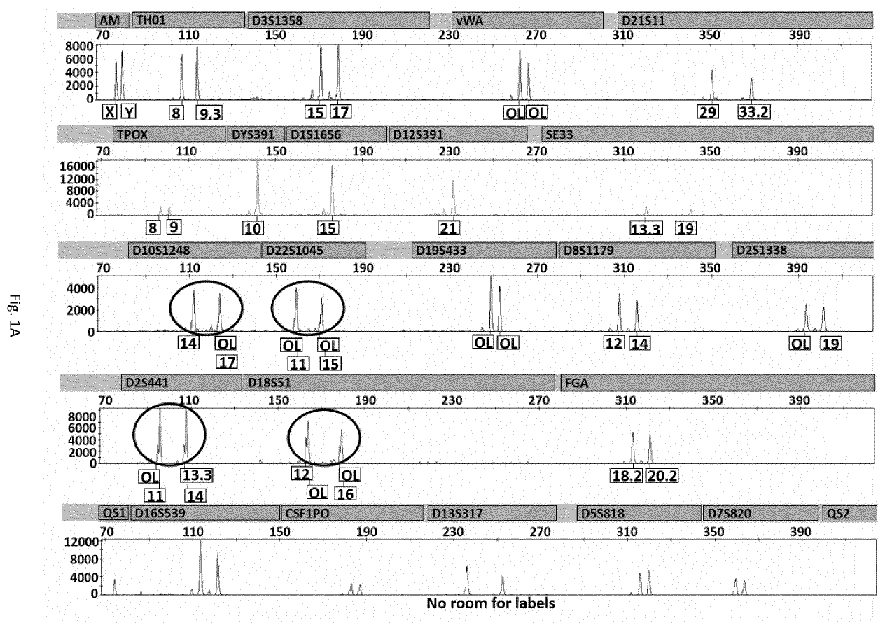

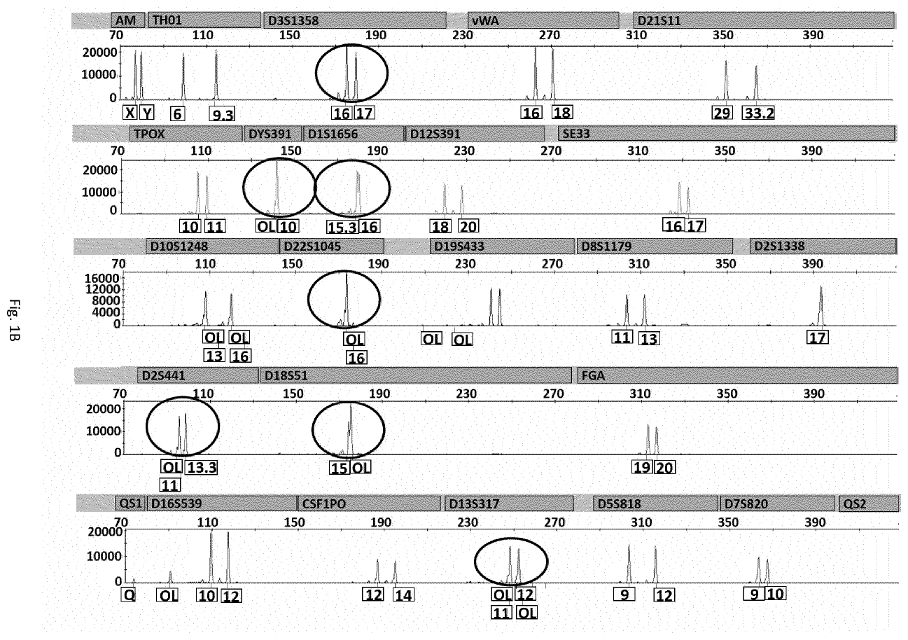

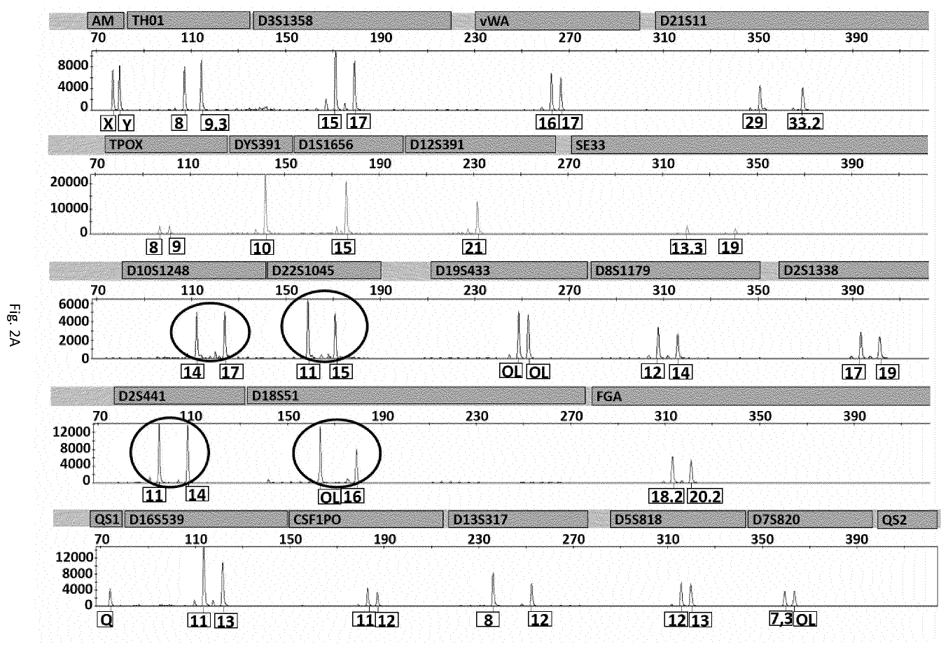

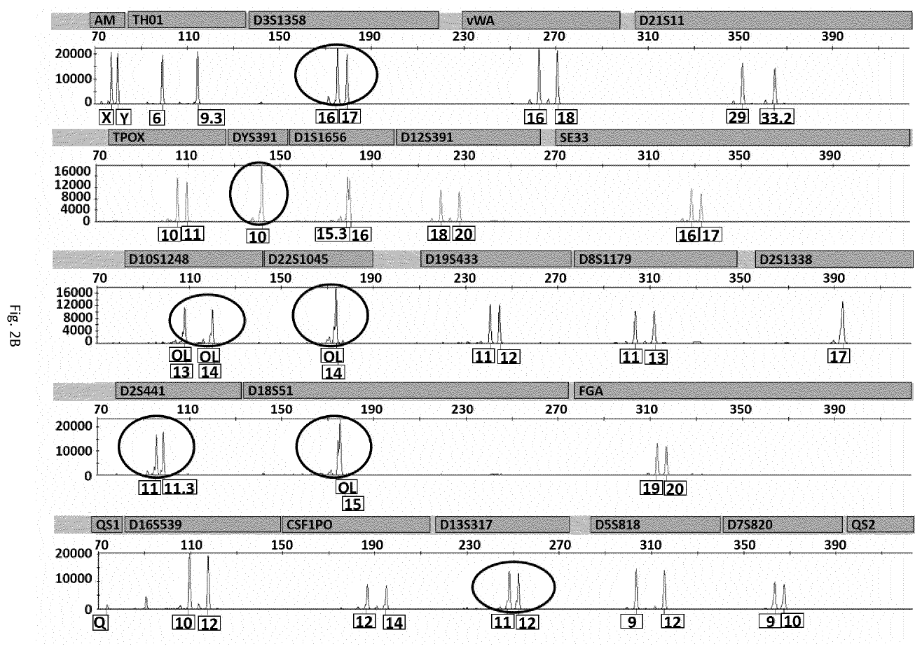

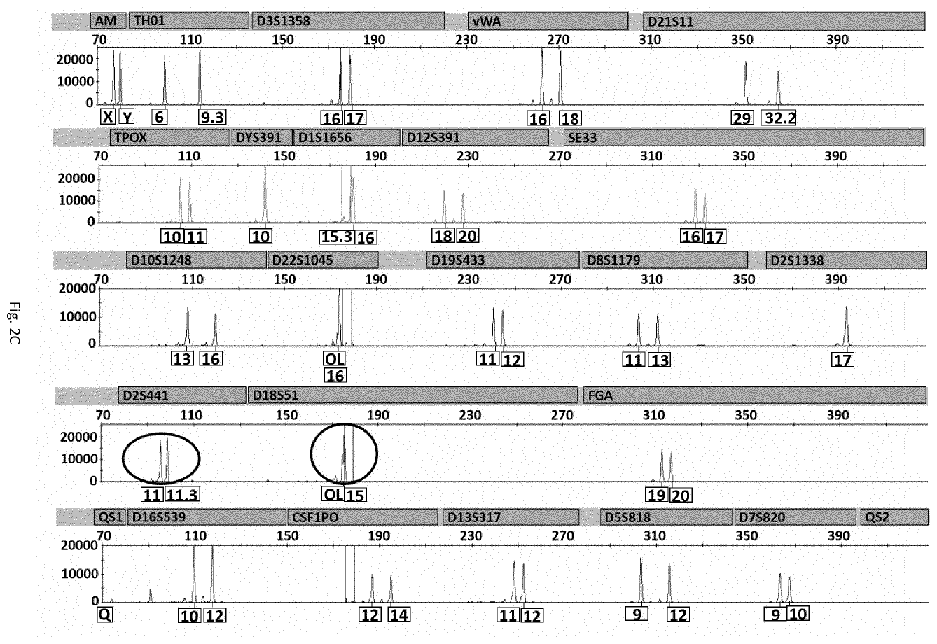

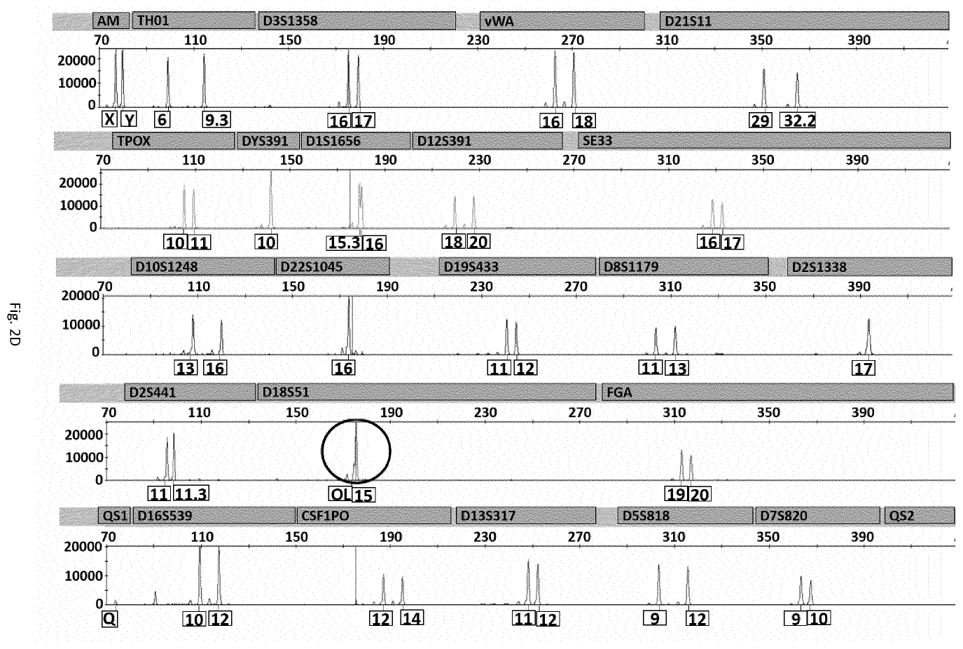

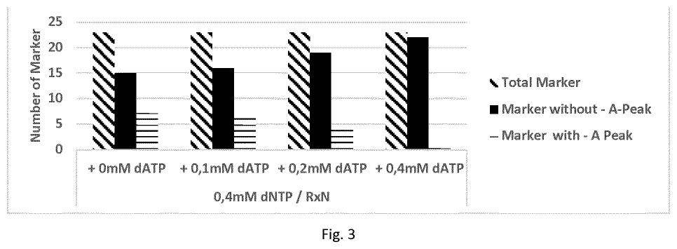

### DETAILED DESCRIPTION OF THE INVENTION

Here, the inventors describe a composition and a method for amplifying, analyzing and typing polymorphic DNA fragments, particularly minisatellite, microsatellite or STR DNA fragments in a fast, reliable and cost-effective way.

The present invention effectively solved the problem of split peak formation reported above by using a mix of asymmetric nucleotide concentrations instead of the common equimolar concentration of the individual nucleotides (dATP, dCTP, dGTP, dTTP). In particular, the inventors have found that the use of an excess of dATP over dCTP, dGTP, dTTP promotes the generation of an A overhang so that split peak formation during PCR can be successfully prevented.

In a first aspect, the present invention provides a composition for performing an amplification reaction of a nucleic acid template, the composition comprising
a. a buffer,
b. a DNA polymerase,
c. one or more primers and
d. a mixture of deoxynucleotides (dNTPs),

wherein the mixture of dNTPs comprises a higher dATP concentration than that of either dGTP, dCTP or dTTP.

In one embodiment, the concentration of dATP is between 1,5-fold and 2,5-fold, preferably 1,8-fold and 2,2-fold and most preferably between 1,9-fold and 2,1-fold in excess over the concentration of dGTP, dCTP or dTTP.

As used herein, the term “dNTPs” refers to deoxyribonucleoside triphosphates. Non-limiting examples of such dNTPs are dATP, dGTP, dCTP, dTTP, dUTP, which may also be present in the form of labelled derivatives, for instance comprising a fluorescent label, a radioactive label, a biotin label. dNTPs with modified nucleotide bases are also encompassed, wherein the nucleotide bases are for example hypoxanthine, xanthine, 7-methylguanine, inosine, xanthinosine, 7-methylguanosine, 5,6-dihydrouracil, 5-methylcytosine, pseudouridine, dihydrouridine, 5-methylcytidine.

As used herein, the term “primer” refers to a molecule comprising a continuous strand of nucleotides sufficiently to permit enzymatic extension during an amplification process such as polymerase chain reaction (PCR). A “set of primers” refers to a plurality of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. The person skilled in the art recognizes that the terms “upstream” and “downstream” or “forward” and “reverse” are not intended to be limiting, but rather provide illustrative orientation of the amplification process. A set of primers is employed to specifically amplify a particular target nucleotide sequence in a given amplification mixture.

As used herein, the term “buffer” refers to a solution which provides a suitable chemical environment for the activity of DNA polymerase. The buffer pH is usually between 8.0 and 9.5 and is often stabilized by Tris-HCl. For Taq DNA polymerase, a common component in the buffer is potassium chloride KCl or MgCl₂, which increased specificity of primer annealing. The person skilled in the art is aware of buffer compositions for successful PCR amplification.

As used herein, the term “DNA polymerase” refers to an enzyme that synthesizes DNA in the 5′-3′ direction from deoxynucleotide triphosphate using a complementary template DNA strand and a primer by successively adding nucleotide to a free 3′-hydroxyl group.

In one embodiment, the amplification reaction is a polymerase chain reaction (PCR)

In another embodiment, the DNA polymerase is a thermostable polymerase.

In another embodiment, the DNA polymerase lacks a 3′-5′ exonuclease activity.

In one embodiment, the DNA polymerase can add non-template nucleotides to the amplified nucleic acid strands.

In one embodiment, the DNA polymerase is selected from the group comprising Taq, Bsu, Bst and Tth. In a preferred embodiment, the DNA polymerase is a Taq polymerase.

The STR analysis requires certain range of DNA template to work successfully. However, it has been observed that a large amount of DNA template favors the formation of 3′ A overhang in the PCR amplicon, which is evidenced in the electropherograms by means of a split peak formation.

In one embodiment, the concentration of the nucleic acid template ranges from 8 pg to 8 ng final per each reaction.

In one embodiment, the nucleic acid template comprises a repetitive element, selected from the group of direct repeats, inverted repeats, microsatellites, minisatellites, tandem repeats and short tandem repeats (STR).

In another embodiment, the repetitive element is a short tandem repeat (STR) sequence.

As used herein, the term “short tandem repeat (STR) sequence” are DNA sequences that occur in non-coding region (locus) wherein two or more nucleotides are repeated, wherein the repeated sequences are directly adjacent to each other, wherein said short tandem repeat (STR) sequences are scattered throughout the human genome and are used to calculate the rarity of that specific profile in the population.

In another embodiment, the short tandem repeat (STR) sequence is selected from the group of loci comprising CSF1PO, FGA, TH01, TPOX, VWA, D3S1358, D5S818, D7S820, D8S1179, D13S317, D16S539, D18S51, D21S11, D1S1656, D2S441, D2S1338, D10S1248, D12S391, D19S433, D22S1045, Amelogenin, SE33.

In one embodiment, each primer used for amplification has a terminal “G” nucleotide at the 5′-end of the primer.

A second aspect of the present invention is directed to a method for amplification of a target sequence, the method comprising the steps of:
a. performing a PCR amplification using the composition according to the first aspect and its embodiments of the present invention, thereby obtaining a PCR product,
b. determining the presence of the target sequence in the PCR product.

As used herein, the term “amplification” refers to methods for copying a target nucleic acid sequence, thereby increasing the number of copies of a selected nucleic acid sequence. The amplification reaction may be exponential or linear. The sequences amplified in this manner form an “amplicon” or “amplification product”. A target sequence may be either DNA or RNA. In the context of the present invention, the target sequence is DNA.

The amplification reaction may be either a non-isothermal or an isothermal. In one embodiment, the amplification reaction is preferably non-isothermal. The non-isothermal amplification method may be selected from the group comprising polymerase chain reaction (PCR), real-time quantitative PCR (rt qPCR) and ligase chain reaction (LCR). In the context of the present invention, polymerase chain reaction (PCR) amplification is preferred. Therefore, the term “PCR product” and “amplification product” can be used interchangeably.

The non-isothermal PCR used in the method according to the present invention is characterized by an extended final extension cycle.

The target nucleic acid sequence can be obtained by genomic samples, such as human DNA, animal DNA or microbial DNA (e.g., bacterial, archaeal or fungal), food samples (e.g., animal- or plant-derived), environmental samples (e.g., containing microorganisms).

In one embodiment, the sample subjected to the present method may originate from any of the following specimens comprising whole blood, blood fractions, oral fluids, body fluids, human bioptic tissue or other parts of the human body upon availability for isolation of a genome. As used herein the terms “oral fluids” and “body fluids” refers to fluids that are excreted or secreted from the buccal cavity and from the body, respectively, from which a genome can be isolated. As a non-limiting example, oral and body fluids may comprise saliva, sputum, swab, urine.

The person skilled in the art is aware of suitable method for detection of the PCR product. Examples of such methods to be used in conjunction with PCR include electrophoresis, mass spectroscopy, Sanger sequencing, pyrosequencing, next generation sequencing and the like.

In one embodiment, the target sequence comprises a short tandem repeat (STR) sequence.

In another embodiment, the short tandem repeat (STR) sequence is selected from the group of loci comprising CSF1PO, FGA, TH01, TPOX, VWA, D3S1358, D5S818, D7S820, D8S1179, D13S317, D16S539, D18S51, D21S11, D1S1656, D2S441, D2S1338, D10S1248, D12S391, D19S433, D22S1045, Amelogenin, SE33.

A further advantage of the present invention is that it provides an improved method for detecting STR sequences in a target sequence. Particularly, as the 3′ overhang event affecting PCR products obtained by using a polymerase lacking proof-reading feature, e.g., Taq polymerase, is solved by using the composition disclosed herein, the overall analysis process does not require extensive and costly purification steps.

In a third aspect, the present invention encompasses a primer or set of primers for detecting a target sequence, wherein the primer or each primer in the set of primers has a terminal “G” nucleotide at the 5′-end of the primer.

In a fourth aspect, the present invention provides a kit for STR analysis, the kit comprising:
a. a mixture of dNTPs, the mixture comprising dATP, dGTP, dCTP and dTTP, wherein the concentration of dATP is higher than dGTP, dCTP and dTTP;
b. a set of primers, wherein each primer in the set of primers comprises a 5′-end G;
c. a buffer;
d. a DNA polymerase lacking 3′-5′ exonuclease activity;
e. a nucleic acid template comprising a short tandem repeat (STR) sequence.

### EXAMPLES

### Example 1

### Testing High Levels of DNA Template with Equimolar Concentration of dNTP

PCR amplifications were performed as follows:

Cycler: Veriti

Mix: FRM2.0

System: 24plex QS Primer mix

dNTP conditions as reported in the Investigator 24plex QS handbook (QIAGEN):

+0.4 mM dATP

+0.4 mM dCTP

+0.4 mM dGTP

+0.4 mM dTTP

4 replicates.

Template: 8 ng Flexi Male DNA 5

Cycling conditions as reported in the Investigator 24plex QS handbook (QIAGEN):

| Temperature | Time | |
|---|---|---|
| (° C.) | (sec) | Number of cycles |
| 98 | 30 | 3 |
| 64 | 55 | |
| 72 | 5 | |
| 96 | 10 | 27 |
| 61 | 55 | |
| 72 | 5 | |
| 68 | 120 | — |
| 60 | 120 | — |
| 10 | — | — |

Approach: 10×
75 μl FRM 2.0
25 μl Primer mix
1 μl dATP, dCTP, dGTP or dTTP (100 mM)
100 μl water
each 20 μl MM+5 μl Template (1.63 ng/μl)

As depicted in FIG. 7, it is evident that the use of large amount of DNA template favors the formation of split peaks due to the 3′ A overhang.

### Example 2

### Testing Various Conditions to Eliminate the Split Peaks Occurring at Larger Template Amounts

The following experiment is performed to investigate the effect of testing the excess of dATP over dGTP, dCTP and dTTP along with extension of the final extension steps.

PCR amplifications were performed as follows:

Cycler: 9700

Mix: FRM 2.0

System: 24plex QS Primer Mix

Conditions: normal approach as reported in the Investigator 24plex QS handbook (QIAGEN):

+0.2 mM dNTPs (dGTP, dCTP and dTTP)

+0.4 mM dATP

+50% Taq

+100% Taq

Template: Flexi Male DNA (template amount, see conditions)

Cycling: 24plex QS cycling as reported in the Investigator 24plex QS handbook (QIAGEN) (with prolonged final extension):

| Temperature | Time | |
|---|---|---|
| (° C.) | (sec) | Number of cycles |
| 98 | 30 | 3 |
| 64 | 55 | |
| 72 | 5 | |
| 96 | 10 | 27 |
| 61 | 55 | |
| 72 | 5 | |
| 68 | 900 | — |
| 60 | 900 | — |
| 10 | — | — |

Approach: 18×
135 μl FRM 2.0
45 μl Primer mix
9 μl dNTP mix (10 mM of dGTP, dCTP and dTTP)
1.8 μl dATP (100 mM)
9 μl or 18 μl Taq (15 U/μl)
180 μl water
each 20 μl Mastermix+5 μl Template

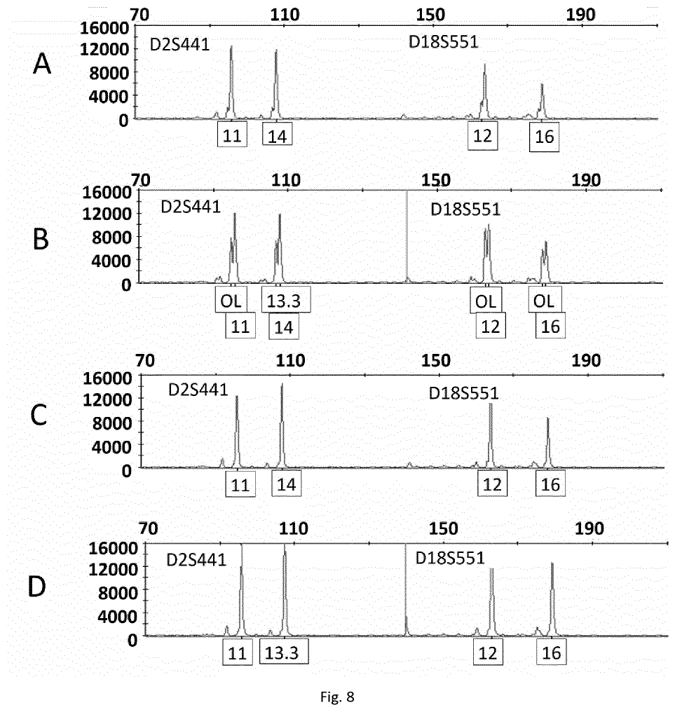

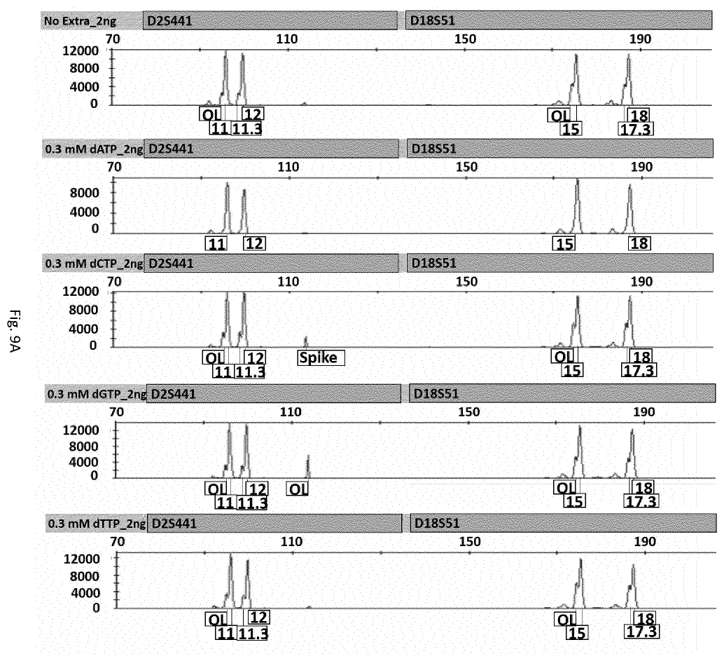

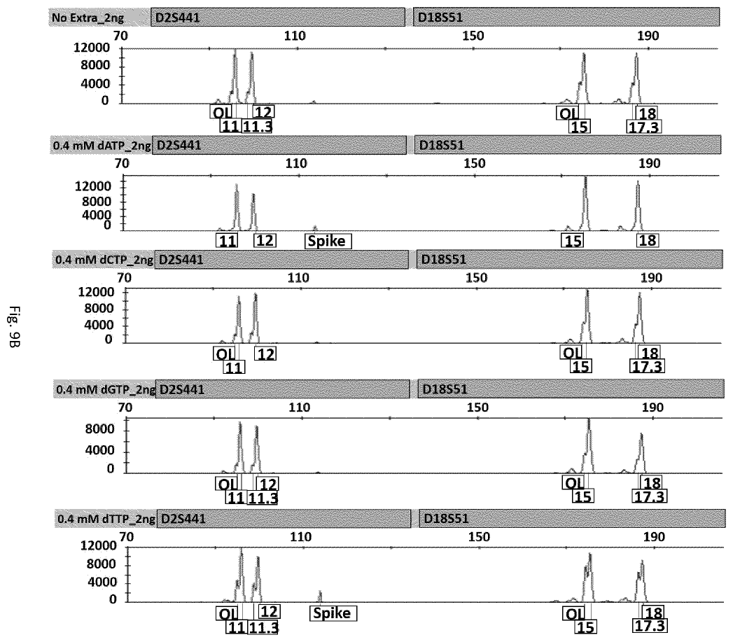

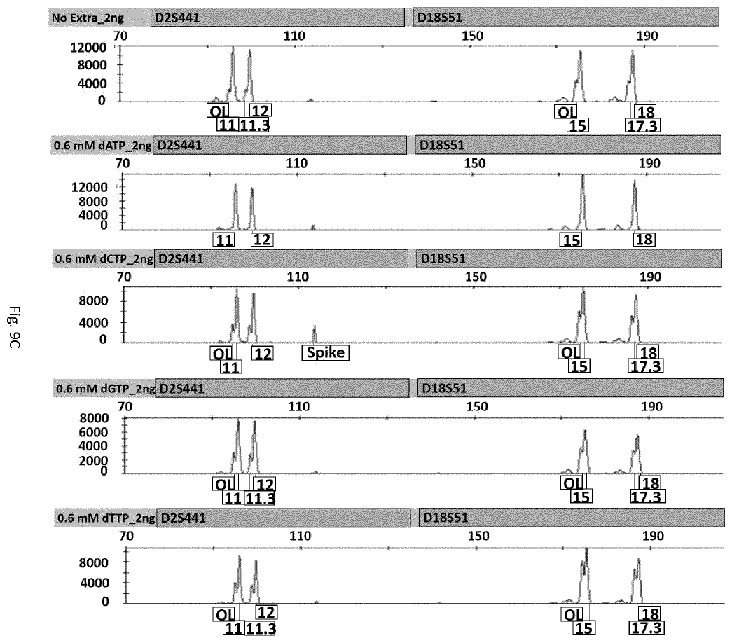

As depicted in FIG. 8, the use of asymmetric dNTP levels, i.e., an excess of dATP over dGTP, dCTP and dTTP along with longer final extension prevents split peak formation completely (even at higher TAQ concentrations which normally show a stronger split peak formation).

It is also evident that the reduction of split peaks phenomena is connected to the concentration of dATP. The alteration of the concentrations of dGTP, dCTP and dTTP shows no improvements in reducing the split peaks phenomena, which do not occur or are significantly reduced with the addition of dATP (see FIG. 7).

### Example 3

### Testing the Excess of dATP with and without Extending the Final Extension Steps

PCR amplifications were performed as follows:

Mix: FRM 2.0

System: 24plex QS

| | | | Final extension at |
|---|---|---|---|
| | DNA template | dATP | 60° C./68° C. time |
| | (ng) | (mM) | (min) |
| Cycler 1 | 8 | — | 2 |
| | 8 | 0.1 | 2 |
| | 8 | 0.2 | 2 |
| | 8 | 0.4 | 2 |

Reactions: 35×25
263 μl FRM 2.0 3.33×
87.5 μl primer 24plex 10×
35 μl DNA 8 ng/μl
139.5 μl H₂O

each well 15 μl MM+10 μl dATP (dilution below) or H₂O (negative control w/o extra dATP)

0 mM dATP extra, 10 μl H₂O

0.1 mM dATP extra Dilution→0.25 mM, 10 μl each reaction

0.2 mM dATP extra Dilution→0.5 mM, 10 μl each reaction

0.4 mM dATP extra Dilution→1 mM, 10 μl each reaction

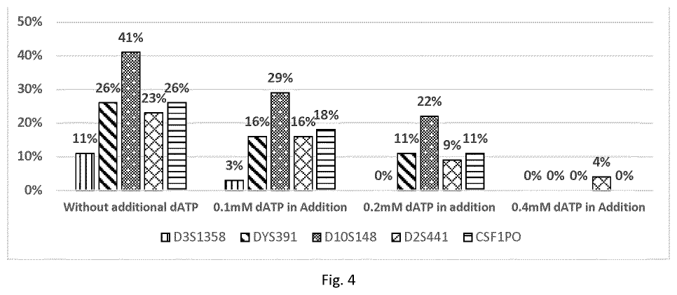

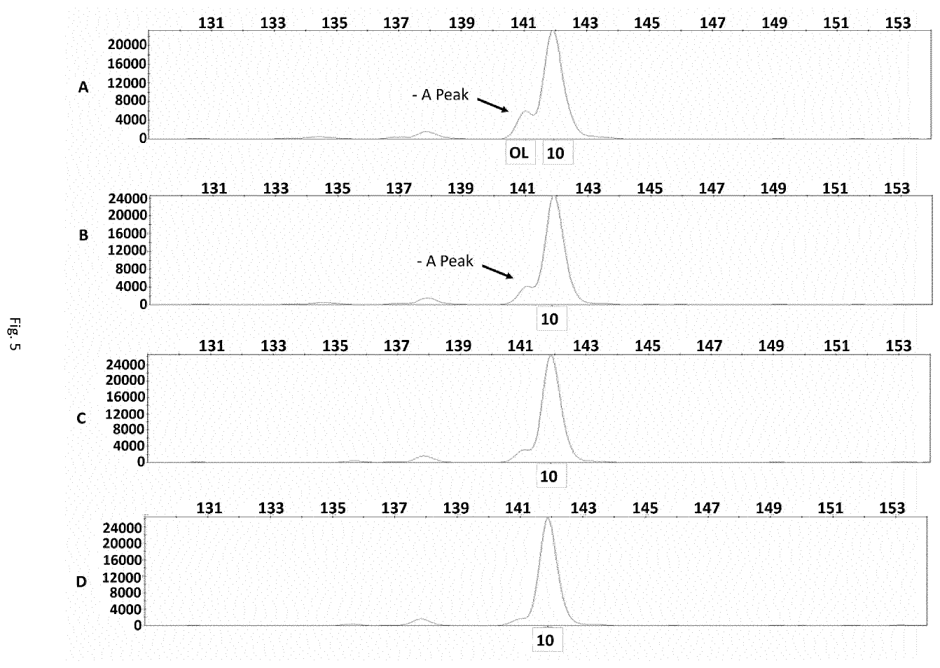

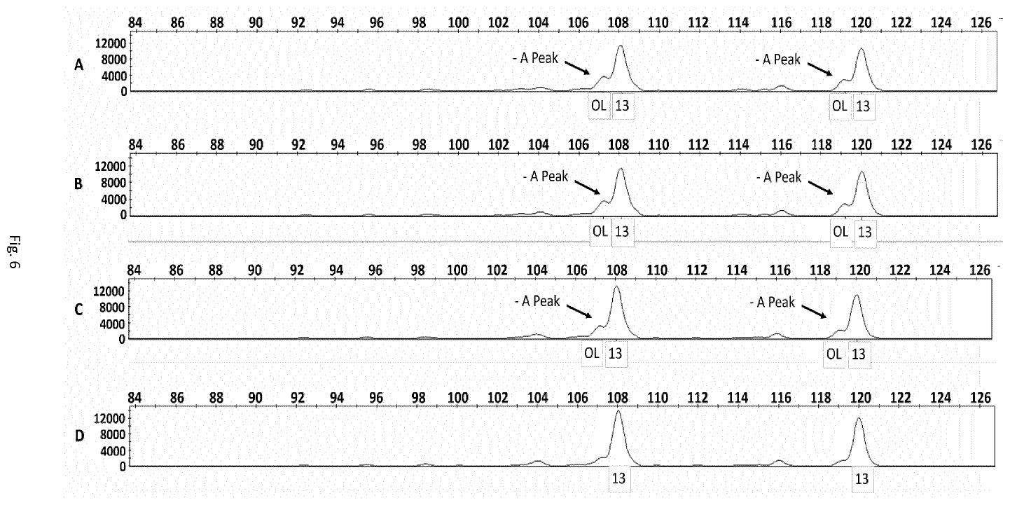

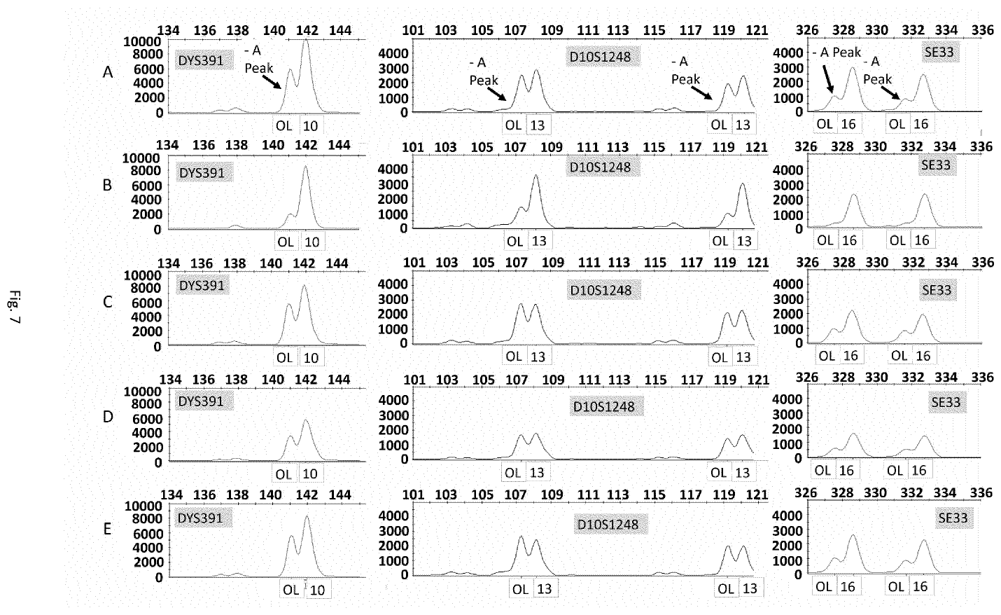

0.2 mM dATP for 5 min at 60° C. and 5 min at 68° C. is sufficient to significantly reduce the −A peaks at high level of DNA template. It is also observed that this protocol does not lead to the formation of +A peaks (see FIG. 4).

Cycling:

| Temperature | Time | |
|---|---|---|
| (° C.) | (sec) | Number of cycles |
| 98 | 30 | 3 |
| 64 | 55 | |
| 72 | 5 | |
| 96 | 10 | 27 |
| 61 | 55 | |
| 72 | 5 | |
| 68 | 120 | — |
| 60 | 120 | — |
| 10 | — | — |

### Example 4

### The Effect of Split Peak Elimination is Specific to an Excess of dATP

The following experiment was performed in order to investigate whether the effect of split peak elimination is specifically achieved by an excess of dATP or if an excess of ether dCTP, dGTP or dTTP leads to the same results.

PCR amplifications were performed as follows:

Cycler: ABI GeneAmp 9700

Master Mix: Fast Reaction Mix (FRM) 2.0

System: 24plex QS Primer Mix

Conditions: normal approach as reported in the Investigator 24plex QS handbook (QIAGEN; Cat. No.: 382415):

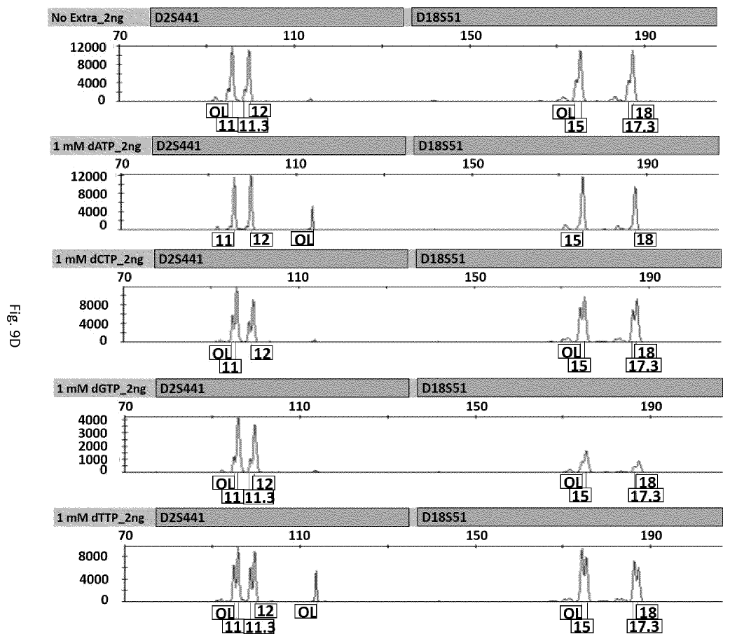

+0.3 mM dATP or dGTP or dCTP or dTTP (FIG. 9A)

+0.4 mM dATP or dGTP or dCTP or dTTP (FIG. 9B)

+0.6 mM dATP or dGTP or dCTP or dTTP (FIG. 9C)

+1.0 mM dATP or dGTP or dCTP or dTTP (FIG. 9D)

Template: Control DNA 9948 (5 ng/μl) (QIAGEN; Cat. No.: 386041); diluted to 200 pg/μl.

Cycling: 24plex QS cycling as reported in the Investigator 24plex QS handbook (QIAGEN):

| Temperature | Time | |
|---|---|---|
| (° C.) | (sec) | Number of cycles |
| 98 | 30 | 3 |
| 64 | 55 | |
| 72 | 5 | |
| 96 | 10 | 27 |
| 61 | 55 | |
| 72 | 5 | |
| 68 | 120 | — |
| 60 | 120 | — |
| 10 | — | — |

Approach: 96×25 μl PCR reactions
720 μl FRM 2.0
240 μl Primer mix 10×
480 μl H2O

79.5 μl (=5,3 reactions) of above mentioned mix+one of 1-5:
1) 26.5 μl H2O (=equimolar dNTPs; “No_Extra”);
2) 3.975 μl of 10 mM dATP or dTTP or dCTP or dGTP (final concentration 0.3 mM)+22.5 μl H2O
3) 5.3 μl of 10 mM dATP or dTTP or dCTP or dGTP (final concentration 0.4 mM)+21.2 μl H2O
4) 7.95 μl of 10 mM dATP or dTTP or dCTP or dGTP (final concentration 0.6 mM)+18.6 μl H2O
5) 13.25 μl of 10 mM dATP or dTTP or dCTP or dGTP (final concentration 1.0 mM)+13.25 μl H2O

Each reaction/well was performed with 20 μl mastermix+5 μl of diluted template DNA (=1 ng). Non-template control reactions were performed as well in order to exclude possible DNA contaminations in the mastermix. All reactions were run in duplicates.

As depicted in FIG. 9, only an excess of dATP led to an elimination of split peaks whereas an excess of either dTTP, dCTP or dGTP had no effect on the formation of split peaks.

1. A composition for performing an amplification reaction of a nucleic acid template, the composition comprising
a. a buffer,
b. a DNA polymerase,
c. one or more primers and
d. a mixture of deoxynucleotides (dNTPs),
wherein the mixture of dNTPs comprises a higher dATP concentration than that of either dGTP, dCTP or dTTP.

2. The composition according to claim 1, wherein the concentration of dATP is between 1,5-fold and 2,5-fold, preferably 1,8-fold and 2,2-fold and most preferably between 1,9-fold and 2,1-fold in excess over the concentration of dGTP, dCTP or dTTP.

3. The composition according to claim 1, wherein the amplification reaction is a polymerase chain reaction (PCR).

4. The composition according to claim 1, wherein the DNA polymerase lacks a 3′-5′ exonuclease activity.

5. The composition according to claim 1, wherein the DNA polymerase is a thermostable polymerase.

6. The composition according to claim 1, wherein the DNA polymerase can add non-template nucleotides to the amplified nucleic acid strands.

7. The composition according to claim 1, wherein the DNA polymerase is a Taq polymerase.

8. The composition according to claim 1, wherein the concentration of the nucleic acid template ranges from 8 pg to 8 ng.

9. The composition according to claim 1, wherein the nucleic acid template comprises a repetitive element, selected from the group of direct repeats, inverted repeats, microsatellites, minisatellites, tandem repeats and short tandem repeats (STR).

10. The composition according to claim 9, wherein the repetitive element is a short tandem repeat (STR) sequence.

11. The composition according to claim 10, wherein the short tandem repeat (STR) sequence is selected from the group of loci comprising CSF1PO, FGA, TH01, TPOX, VWA, D3S1358, D5S818, D7S820, D8S1179, D13S317, D16S539, D18S51, D21S11, D1S1656, D2S441, D2S1338, D10S1248, D12S391, D19S433, D22S1045, Amelogenin, SE33.

12. A method for amplification of a target sequence, the method comprising the steps of:
a. performing a PCR amplification of a target sequence using the composition according to any of the claims 1 to 11, thereby obtaining a PCR product,
b. determining the presence of the target sequence in the PCR product.

13. The method according to claim 12, wherein the target sequence comprises a short tandem repeat (STR) sequence.

14. The method according to claim 12, wherein the PCR amplification is a non-isothermal PCR.

15. A kit for STR analysis, the kit comprising:
a. a mixture of dNTPs, the mixture comprising dATP, dGTP, dCTP and dTTP, wherein the mixture of dNTPs comprises a higher dATP concentration than that of either dGTP, dCTP or dTTP;
b. one or more primers, wherein each primer used for amplification has a terminal “G” nucleotide at the 5′-end of the primer;
c. a buffer;
d. a DNA polymerase;
e. a nucleic acid template comprising a short tandem repeat (STR) sequence.
